(19) United States
(12) Patent Application Publication
(10) Pub. No.: US 2012/0245586 A1
(43) Pub. Date: Sep. 27, 2012

(54) Bone Drill

(75) Inventors: Petri Lehenkari, Oulu (FI); Arto Nykänen, Oulu (FI)

(73) Assignee: OULUN YLIOPISTO, Oulu (FI)

(21) Appl. No.: 13/425,492

(22) Filed: Mar. 21, 2012

(30) Foreign Application Priority Data
Mar. 21, 2011 (FI) U20110105
Mar. 21, 2012 (FI) PCT/FI2012/050283

(51) Int. Cl. A61B 17/16

(52) U.S. Cl. 606/80

(57) ABSTRACT

Bone drill (100) for medical operations. In the bone drill, there is an elongated drill component (101), which is manufactured from a superelastic material, and an essentially straight drill-component shield (103), which is hollow. According to the invention, at the end of the drill-component shield there is a guide arrangement (102) for selecting for the drill component a turning angle (α; β) relative to the drill-component shield, and once the drill component has run through the guide part, the superelastic properties of its material return it to its original shape and the drill component is then (101) is a position, in which it can be used to drill in the direction of the turning angle (α; β).

The invention relates to a bone drill for medical operations, in which bone drill there is an elongated drill component, which is manufactured from a superelastic material, and an essentially straight drill-component shield, which is hollow.

In many medical operations holes or channels in bone are required. These are used, for example, for inserting screws, for removing a damaged or diseased part of a bone, or for inserting a medicament or implant in a bone. There are many different bone drills for these purposes.

Generally, it is difficult to guide a drill inside a bone. It is even more difficult if a curve must be made in the channel or hole.

For drilling, drills have been developed, in which components made from a superelastic material are used as the drilling part. A piece made from a superelastic material seeks to return to its original shape. One material widely used in medical devices is nitinol, an alloy of titanium and nickel.

U.S. Pat. No. 6,068,642 discloses a bone drill, in which a superelastic drill component is used. In the drill, there is a tubular support part for guiding the drill component, and the drill component runs inside this support part. At the outer end of this support part there is a bend, and the drill component bends as it runs through it. The support part runs inside a larger external tube, in the sides of which are openings. The end of the support part is brought to the location of such an opening and the drill component can be guided through this opening. Because the superelastic material returns to its shape, the drill component proceeds in a straight line once it has exited the hole. In this case, the external tube must be quite thick, to allow the support part to move inside it. In addition, in practice in this case only a hole that is at right angles to the main channel can be obtained. Further, in such a solution even small movements of the drill can cause the drill component to jam. In addition, the drilling component acts as an abrading and cutting blade.

A surgical drilling device, which is used, for example, for draining tissue inside the bone, is known from international application publication WO 2003/101308. In it, there is a superelastic drilling and suction component. The drilling component, which comes out of a straight shield component, drills a channel or is pushed into the tissue. The drilling and suction component can be shaped into a curve. When it comes out of the straight shield component, its superelastic properties cause it to return to its curved shape. The direction in which it will start curving can be selected by rotating the drilling and suction component.

It is difficult to drilling actual holes using this method, because when it returns to its original curved shape the arm of the drilling and suction component catches on the walls of the already drilled hole and the rotating arm will then begin to wear the wall of the hole.

The invention is intended to create a solution, by means of which it is possible to significantly reduce the detriments and drawbacks relating to the prior art.

The basic idea of the invention is that a superelastic material is used in the drill component of the bone drill and there is a guide arrangement at the end of an essentially straight drill-component shield, which turns the drill component, typically to an angle deviating from the axial direction of the drill-component shield, when it exits from inside the drill-component shield. As the original shape of the drill component is essentially straight, its returns to this position and can drill in the direction in which it was turned using the guide arrangement.

The aims according to the invention are achieved by means of a bone drill, which is characterized by what is stated in the independent Claim. Preferred embodiments of the invention are presented in the dependent Claims.

Considerable advantages are achieved by means of the invention. Thus, by means of the present bone drill, the drilling direction can be easily altered. In addition, the invention has the advantage that holes can be drilled in several directions from the same drilling channel.

Further, the invention permits the precise control of drilling. The construction of the invention also reduces the risk of the drill component jamming and permits the use of a greater axial force and a blade that displaces and compacts bone.

The invention also has the advantage that, by means of a drill according to it, it is possible to drill into locations that are extremely difficult to reach using conventional means.

A further advantage of the invention is that it can be used to reduce the number of drillings, which both accelerates operations and reduces the strain on the subject.

In the device according to the invention, the superelastic metal-alloy bit can, for example, be made to bend in the drill channel, making it possible to direct the drilling inside the bone. Because the device is equipped with a drill-bit guide, the slant of the drill bit can be set freely within a preset range, which is, for example, 5-45 degrees, relative to the longitudinal axis of the drill bit, i.e. the axial direction.

With the aid of the invention, bone drilling can be done safely, in a controlled and correctly-oriented manner.

In the following, the technology being presented will be examined with the aid of a detailed description, with reference to the accompanying drawings.

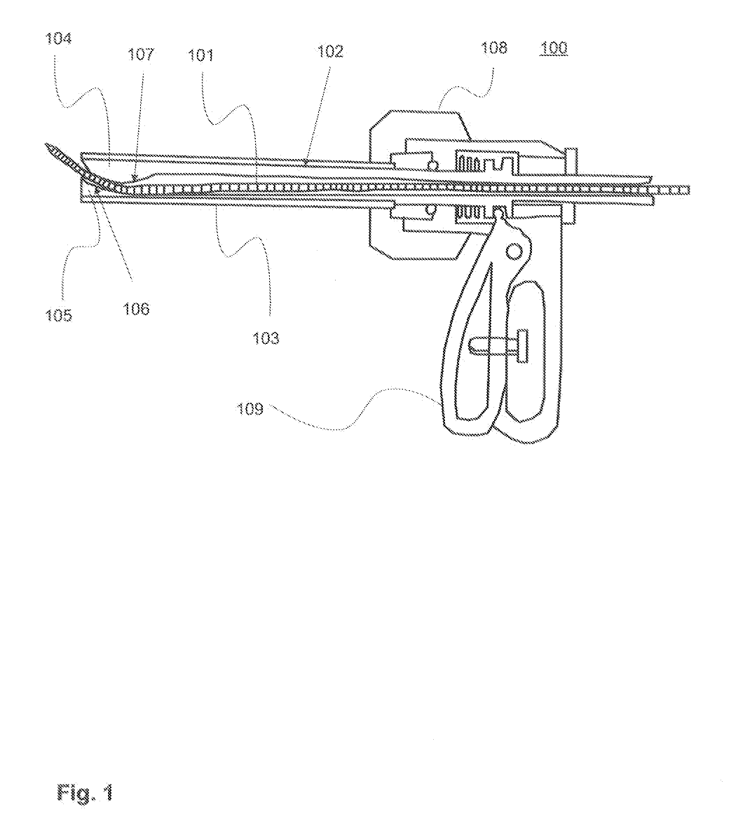

FIG. 1 shows, by way of example, a side cross-section of a bone drill according to a first embodiment,

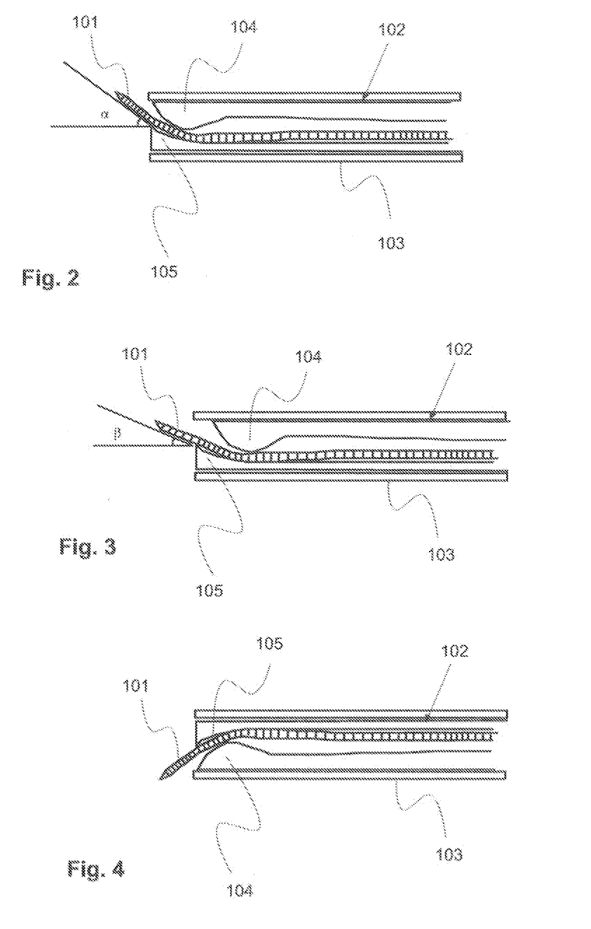

FIG. 2 shows an example of the guiding of the drill component of the bone drill according to FIG. 1,

FIG. 3 shows a second example of the guiding of the drill component of the bone drill according to FIG. 1,

FIG. 4 shows a third example of the guiding of the drill component of the bone drill according to FIG. 1,

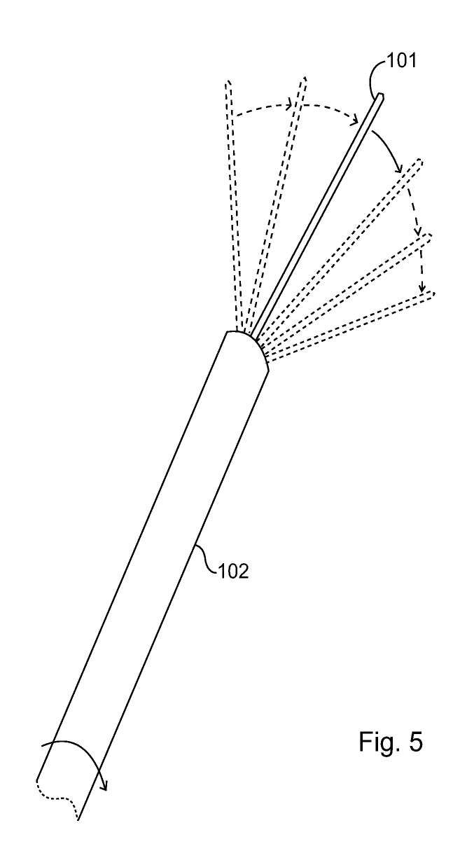

FIG. 5 shows a perspective view of how, by rotating the bone drill around its longitudinal axis, the drill bit can be used to drill holes in several directions, and

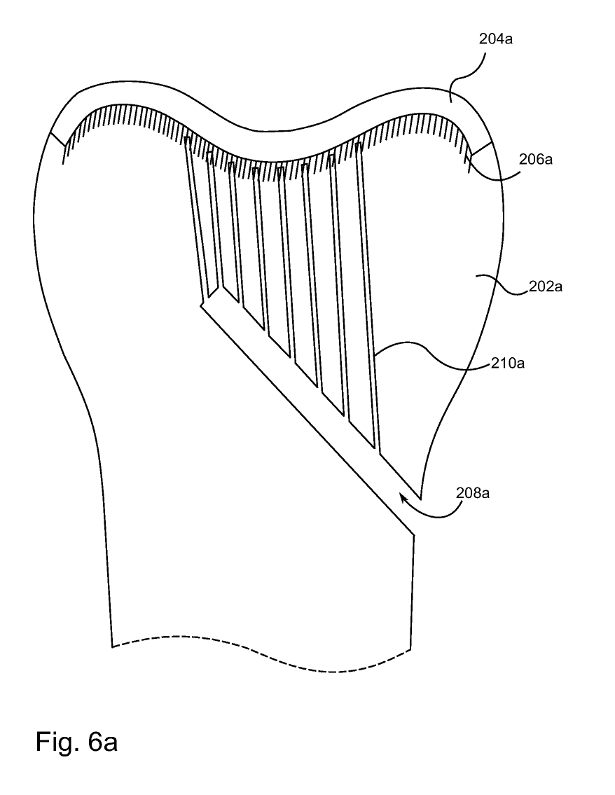

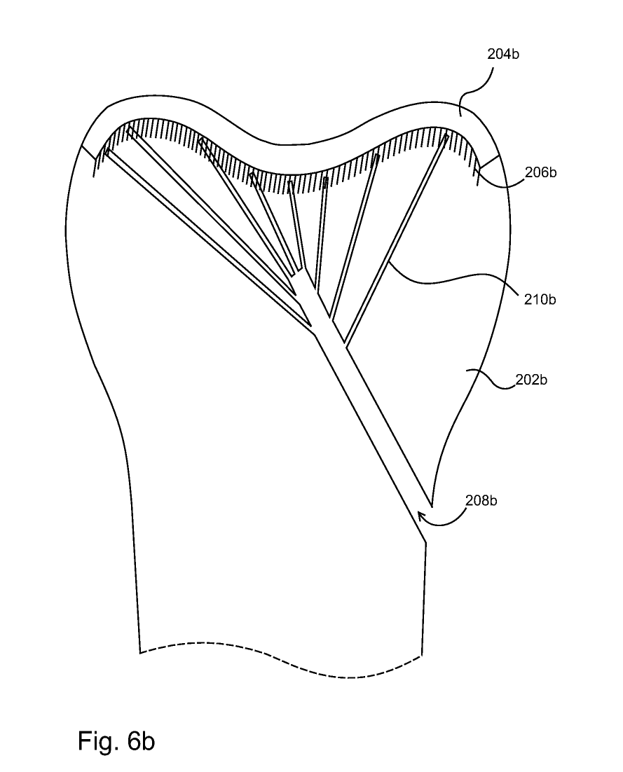

FIGS. 6a and 6b show a side view of a treatment implemented as a drilling into the subchondral bone, in which a first elongated drill channel is formed in a femur, from which second drill channels branch out and extend into the bone layer located under the cartilage layer, and with the aid of which the renewal of the cartilage layer can be promoted.

As stated above, the present bone drill is intended mainly for medical operations. Particularly, the bone drill can be used to create a drill channel in bone of biological origin, such as a bone of a living mammal.

In the bone drill, there is typically an elongated drill component, which is manufactured from a superelastic material, and an essentially straight drill-component shield, which is hollow. The shield surrounds the drill component. The elongated drill component has a longitudinal axis.

In the first embodiment, the drill-component shield is tubular and open at the end. In the present case, the term open “end” of the tubular shield refers to its “distal end”, or the point of the shield. This is the end of the shield that is farthest from the user of the drill. In particular, the end in question faces away from the user. Most appropriately, the open portion of the end in question faces in a direction 180 degrees in the opposite direction compared to the proximal end of the tubular shield (i.e. the end closest to the user).

In a preferred embodiment, the shield is equally thick over the entire area of the tubular part, including the end, i.e. it has a constant diameter throughout.

The distal end is arranged to be pushed into a drill hole or channel made in the bone, which means that, at it, the external diameter of the drill-component shield is smaller than the internal diameter of the drill hole or channel in question. The external diameter of the bone drill is preferably smaller than the internal diameter of the drill hole or channel over the entire length of the tubular shield.

The elongated, straight, and hollow drill-component in question is, in its interior, shaped to bend the drill component rotating inside it, as will be described in greater detail below.

Thus, in the bone drill, the drill component is arranged to be moved in such a way that a rotating movement is created in it and it can be pushed out from inside the drill-component shield, when it drills a hole in the bone. In the end of the drill-component shield there is a guide arrangement, in one preferred embodiment a fixed guide arrangement, through which the drill component runs. The guide arrangement is used to select the turning angle of the drill component relative to the longitudinal axis of the drill component. After running through the guide arrangement, the drill component exits the device through the open surface of the distal end.

Once the drill component has run through the guide part, the superelastic properties of its material are able to return it to its original shape and the drill component is then in a position, in which it can be used to drill in the direction of the turning angle.

“Turning angle” refers to the angle, which the part of the drill component that has run through the guide part forms relative to the drill component's original longitudinal axis. In one embodiment, the turning angle is about 5-45 degrees relative to the drill bit's longitudinal axis, i.e. the axial direction.

“Drilling direction” refers to the direction in which the drilling proceeds, i.e. the direction in which the drill component travels in the material, particularly bone, being drilled.

In one embodiment of the bone drill, the guide arrangement consists of a single component and is attached to the very end of the drill-component shield. It can be fixed, in which case it creates a fixed turning angle. A curved tubular construction, for example, is suitable for this purpose.

In a second embodiment of the bone drill, the guide arrangement can be changed in order to change the turning angle. The turning angle is then specific to each guide arrangement.

In a third embodiment of the bone drill, the guide arrangement is inside the drill-component shield and, in the guide arrangement, there is a slide component and a counter-component, and the drill component is arranged to run between the slide component and the counter-component, in such a way that they alter the turning angle of the drill component.

In a fourth embodiment of the bone drill, the guide arrangement comprises or consists of a single component and can be changed in order to change the turning angle.

In a fifth embodiment of the bone drill, the guide arrangement comprises or consists of at least two components and a slide component and counter-component are shaped in the guide arrangement in order to guide the drill component, in such a way that the slide component and counter-component are is different parts of the guide arrangement. In this embodiment, typically that part of the guide arrangement, in which the slide component is, can be moved relative to the counter-component, allowing the turning angle of the drill component running between the slide component and the counter-component to be altered.

In a sixth embodiment of the bone drill, the guide arrangement can be moved around its axis inside the drill-component shield, in order to change the direction of the drill component relative to the axis of rotation of the guide arrangement. I.e., by rotating the guide arrangement it is possible to change the direction in which the drill component exits from inside the drill-component shield.

In a seventh embodiment of the bone drill, there is a slide surface in the slide component and a counter surface in the counter-component, and the said surfaces are arranged to bend the drill component running between them, and the positions of the surfaces relative to each other determine the magnitude of the bending of the drill component.

The present solution is suitable for various therapeutic treatments and surgery. Particularly with its aid one or several drill channels can be formed in a desired direction in a bone, the longitudinal direction of which can differ from the drill component's original drilling direction.

Drilling is typically performed in such a way that first of all a first drill channel, which hereinafter is also referred to as a lead-in channel, with an internal diameter that is somewhat larger that the external diameter of the drill shield, is formed in the bone using, for example a tubular drill. The present drilling device is taken into this first drill channel and is pushed to the desired depth, after which a drill bit rotating around its longitudinal axis is led out through the end and, with its aid, a smaller, second drill channel is formed in the adjacent bone. This drilling direction typically differs from the longitudinal direction of the first drill channel and is the same as the ‘turning direction’ of the drill component. The direction can be selected by means of the drill device's guide arrangement.

Thus, through one and the same drill channel (the lead-in channel) it is possible to bring to the bone a drill bit, the drilling direction of which at the end of the drill channel can differ from the original.

For example, the area at the end of a femur can be drilled full of radial holes from a single channel. This is possible by rotating the straight sleeve part, which surrounds the drill component concentrically, around the longitudinal axis of the drill component (the guide arrangement, i.e. the bender, being inside the straight part).

Thus, the lead-in channel of the drilling device is the greatest trauma in the bone and drilling of the target area can be performed mini-invasively. A corresponding principle applies to other bones too.

With the aid of a fluid-tight insert attached to a separate syringe, a biologically or physiologically active substance, such as a medication (e.g., bisphosphonate affecting osteoclasts), stem cells, or a local anaesthetic can be injected into the drill channels made with the present bone-drill. In addition to the treatment of osteonecrosis, the invention can be used in many other applications, for example, in joint-surface drilling (so-called Back's drilling) and in arthrodesis. With its aid, drilling can be performed more physiologically from the bone side (subchondral-bone drilling). One example of such an application is, for example, the drilling on unossified bone through the trabecular bone in intramedullary nailing.

The present device can also be used in retrograde osteochondritis drilling and osteoarthritis-decompressions.

In the following, the solutions according to the drawings are examined in greater detail:

FIG. 1 shows a side cross-section of an example of a bone drill 100 according to the invention.

In the bone drill, there is a drill component 101, a drill-component shield 103, a guide arrangement 102, a drill-component shield attachment arrangement 108, and a control part 109. For reasons of simplicity, normal bone-drill components and functionalities have been omitted from the figure.

The drill component 101 is an elongated construction with a circular cross-section, which is at least partly manufactured from a superelastic material. One possible material, which is widely used in medical equipment, is nitinol (NiTi), an alloy of titanium and nickel. In this preferred embodiment, the blade part has a compacting effect and drilling is based on the rapid rotation of the blade and on axial force. The drilling effect of the solution thus differs from that of, for example, the bone drill according to U.S. Pat. No. 6,068,642, referred to in the preamble.

Other materials too are possible. What is essential is that the part of the drill component manufactured from a superelastic material, particularly from a superelastic metal-alloy material, can be bent, or otherwise shaped, and, when the shaping force is removed, the drill component returns to its original shape.

In the drill component, there is a point, by means of which the drill component drills a hole, when the drill component is rotated. The drill component is rotated by a motor. In addition, the drill component protrudes from inside the bone drill and is refracted into the bone drill. This is implemented by means of some conventional arrangement.

The thickness of the drill component is typically about 0.1-10 mm, particularly about 0.5-5 mm, most suitably about 0.8-2 mm.

The drill-component shield 103 is a hollow elongated tube, in which there is a first end and a second end, which are both open. The first end is the end that is inside the bone when the bone drill is used and the second end is attached to the other structures of the bone drill.

The drill-component shield is moved in a channel made in the bone and, with its aid, the drill component is taken to the drilling location. The drill component can be moved inside the drill-component shield. The first end of the drill-component shield can be shaped in such a way as to facilitate its movement in the channel and, in addition, there can be shaping or shapings to facilitate the movement and guiding of the drill component. For example, the walls of the first end of the drill component can be shaped with a bevel on either the internal or external surface, or on both.

The drill-component shield 103 is attached to the bone drill 100 by means of the drill-component shield attachment arrangement 108. The attachment arrangement permits the shield to be detached, for example, for cleaning or replacement. The operation of the bone drill is controlled by the control part 109. Inside the drill-component shield 103 is the guide arrangement 102. The guide arrangement is used to guide the drill component 101. The guide arrangement can consist of one or several components. The guide arrangement bends the drill component in such a way that it exits from inside the drill-component shield from its first end at an angle that is referred to as the turning angle. Because the drill component is manufactured from a superelastic material, it returns to its original shape. If the un-bent drill component is straight, the drill component exiting from inside the drill-component shield and bent by the guide arrangement will also return to become straight. The drill component will thus have changed its direction. This direction deviates from the direction in which the drill-component shield moves. The angle of this direction, i.e. the turning angle, can be altered by adjusting the guide component or changing its direction. Thus, the drill component can be used to drill in directions differing from that of the channel, in which the drill-component is. In addition, the guide component can rotate around its longitudinal axis inside the drill-component shield. It is therefore possible to change the radial direction of the drill component relative to the channel in which the drill-component shield is.

The guide component can be detached from the bone drill 100, for example, for cleaning, or to change it.

In the guide arrangement 102, there is a slide component 104 and a counter component 105. In the slide component, there is a slide surface 107 and in the counter component there is a counter surface 106, which surfaces are curved, i.e. there are no corners or folds in them, on which the drill component could catch. These components are set in such a way as to form a space, between the slide surface and the counter surface, which has a shape such that it bends the drill component 101, which is pushed through the guide arrangement. This bending is selected to be such that the turning angle of the drill component is that, in which it is desired that the drill component will drill. The drill component is pushed through the space formed by the slide surface and the counter surface at such a speed that the superelastic properties of the material of the drill component are able to return the drill component to the position in which it is desired to drill. This is preferably the original position of the drill component. The counter component is closer than the slide component to the first end of the drill-component shield 103. The counter component is preferably in the opening of the first end, or in its immediate vicinity.

When the guide arrangement 102 consists of a single part, the space between the slide surface 107 and the counter surface 106 remains unchanged. This directs the drill component running through the guide part in a direction that remains constant. However, here too the turning angle can be adjusted by moving the guide arrangement inside the drill-component shield 103, in such a way that the internal wall of the drill-component shield becomes part of the guide arrangement, i.e. the internal wall too bends the drill component. When the guide arrangement is retracted into the drill-component shield, the internal wall bends the drill component in such a way that the turning angle of the drill component is smaller than the turning angle obtained by using only the guide arrangement.

The guide arrangement 102 can also consist of two or several components. The slide component 104 and the counter component 105 can then be in different parts of the guide system. There is also an embodiment, in which the slide component and the counter component are in the same part, and in that case the guide arrangement functions essentially in the same way as the guide arrangement described above and consisting of a single part.

If the slide component and the counter component are in different parts, the guide arrangement is formed in such a way that the slide component can be moved relative to the counter component. When the slide component is moved, the space formed between the slide surface and the counter surface changes. The bending force directed by this space on the drill component 101 then also changes and the turning angle of the drill component changes. For example, in the case according to FIG. 1, when the slide component is moved deeper into the drill-component shield 103, the drill component is bent less and the turning angle decreases. In addition to this, the entire guide arrangement can still be moved inside the drill-component shield. The movement of the guide arrangement and the guide arrangement components is performed in some known manner.

FIG. 2 shows an example of the guiding of the bone drill shown in FIG. 1. In this case, inside the drill-component shield 103 there is a guide arrangement 102, in which there are at least two components and the slide component 104 and the counter component 105 are in different parts. The drill component 101 running through the guide arrangement bends between the slide component and the counter component and, when it exits from the opening of the first end of the drill-component shield, it has a turning angle α.

FIG. 3 shows a second example of guiding the bone drill shown in FIG. 1. In this case, the slide component 104 has been moved slightly more deeply into the drill-component shield 103 and the drill component 101 now bends between the slide component and the counter component 105, in such a way that it has a turning angle 13, which is smaller than the turning angle α in FIG. 2. In this way, the drill component's turning angle can be altered.

FIG. 4 shows a third example of guiding the bone drill shown in FIG. 1. The guide arrangement 102 is inside the drill-component 103 in otherwise the same position as in FIG. 2, but the guide arrangement has been turned relative to its longitudinal axis so that the direction, in which the drill component 101 proceeds, differs from that in FIG. 2. The turning angle is the same in FIGS. 2 and 4.

The guide arrangement can also be located in the outer end of the drill-component shield, when it will turn the drill component running through the guide arrangement. This is a curved tubular or trough-like piece. The guide arrangement can be turned by means of some mechanism, or is attached to the outer end of the drill-component shield and can be turned by turning the drill-component shield. In this way, the drill component can be used to drill in different directions from the same channel.

By replacing the guide arrangement with another guide arrangement, which has a different curvature, the drill component's turning angle, and at the same time the drilling angle, can be changed.

FIG. 5 shows how the drill device's drill shield 102 can be turned around its longitudinal axis, so that the drilling direction, i.e. the direction in which the bit 101 proceeds, various according to the turning angle.

The guide arrangement can be arranged permanently in relation to the shield component. It is also possible to arrangement the shield component as a sleeve around the drill component, in such a way that, by turning it 0-360 degrees around the drill component's longitudinal axis, the direction (i.e. “turning angle”) of the drill component can be set.

In both cases, channels diverging radially from a single basic drill channel, i.e. lead-in channel, can be formed.

The drill is suitable for medical, especially surgical operations. The bone drill can be used particularly for treating subjects, mammals, especially people and animals. In a treatment procedure, the drill is typically used to form at least one; most suitably several drill channels in a bone of biological origin, particularly the bone of the subject. The drill is shaped in such a way that it can be taken to a first drill channel formed in the bone beforehand, the internal diameter of which is slightly larger than the external diameter of the tubular drill shield. The drill is taken to a preselected depth in the drill channel in question, after which a second drill channel, differing in direction from the longitudinal direction of the first drill channel, is formed in the wall of the first drill channel. These second channels usually have a diameter that is smaller than that of the first drill channel, their external diameter being typically about 1-90%, particularly about 5-75%, smaller than the external diameter of the first drill channel.

Both the first and the second drill channels have longitudinal axes that are straight, or at least more or less straight. It is possible for there to be a small amount of curvature in the walls of especially the second channels.

FIGS. 6a and 6b show the use according to one preferred embodiment in drilling to be performed from the side of the bone (in a subchondral bone). FIGS. 6a and 6b show how, for example, in a femur 202a; 202b lead-in channels 208a; 208b are first formed, using an as such known ring drill. After this, the present drill is pushed into the channel 208a; 208b, through the outer end of which at regular intervals and at preselected turning angles—the drilling can be controlled using MRSI—a drill bit is introduced, when drill channels 210a; 210b are formed, which extend from the lead-in channel 208a; 208b as far as the bone zone (the subchondral bone) under the cartilage layer 204a; 204b.

FIG. 6a shows a case, in which the drill channels 210a are always at the same angle to the lead-in channel 208a, because a fixed guide device according to FIG. 1 is used in the drill device. For its part, FIG. 6b shows a case, in which the guide arrangement permits a change in angle, so that the angles of the drill channels 210b vary relative to the lead-in channel 208b.

With the aid of the drill channels 210a and 210b, a biologically or physiologically active substance, such as a medication, stem cells, or a local anaesthetic can, if desired be fed into the subchondral-bone layer.

Some preferred embodiments according to the invention are described above. The invention is not restricted solely to the solutions described, but instead the inventive ideal can be applied in numerous ways within the limits set by the Claims.

1. Bone drill for medical operations comprising:
an elongated drill component, which is manufactured from a superelastic material,
an essentially straight drill-component shield, which is hollow,
wherein at the end of the drill-component shield there is a guide arrangement for selecting the turning angle for the drill component relative to the drill-component shield, so that once the drill component has run through the shield component its material's superelastic properties return it to its original shape and the drill component is then in the position, in which it can be used to drill in the direction of the turning angle.

2. Bone drill according to claim 1, wherein the guide arrangement (102) consists of one part and is attached to the outer end of the drill-component shield (103), and it is, for example, a curved tubular construction, or a corresponding fixed arrangement.

3. Bone drill according to claim 2, wherein the guide arrangement can be changed, in order to change the turning angle.

4. Bone drill according to claim 1, wherein the guide arrangement is inside the drill-component shield and in the guide arrangement there is a slide component and a counter component and the drill component is arranged to run between the slide component and the counter component, in such a way that they change the drill component's turning angle.

5. Bone drill according to claim 4, wherein the guide arrangement consists of a single part and it can be changed, in order to change the turning angle.

6. Bone drill according to claim 4, wherein the guide arrangement consists of at least two parts and a slide component and a counter component are shaped in the guide arrangement, in such a way that the slide component and the counter component are in different parts of the guide arrangement, and the part of the guide arrangement in which the slide component is can be moved relative to the counter component, in such a way that the turning angle of the drill component running between the slide component and the counter component can be change.

7. Bone drill according to claim 4, wherein the guide arrangement (102) can be moved around its axis inside the drill-component shield (103), in order to change the direction of the drill component (101) relative to the axis of rotation of the guide arrangement.

8. Bone drill according to claim 4, wherein in the slide component there is a slide surface and in the counter component there is a counter surface, and the said surfaces are arranged to bend the drill component running between them, and the positions of the surfaces relative to each other determine the magnitude of the bending of the drill component.

9. Bone drill according to claim 1, wherein the shield of the bone drill's drill component is arranged as a sleeve around the drill component, so that it can be rotated around the longitudinal axis of the drill component, in order to set the turning angle of the drill component.

10. Bone drill according to claim 1 wherein the shield of the drill component is tubular, it has a proximal end and a distal end, so that the tube is open at least at the distal end, so that the open portion of the distal end points in the opposite direction relative to the proximal end.
